(19) United States
(12) Patent Application Publication
(10) Pub. No.: US 2025/0327003 A1
(43) Pub. Date: Oct. 23, 2025

(54) Intelligent Control System for Solid-State fermentation producing process and Its Applications

(71) Applicants: Jiangnan University, Wuxi (CN); Jiangnan University (Shaoxing) Industrial Technology Research Institute, Shaoxing (CN)

(72) Inventors: Jian MAO, Wuxi (CN); Shuangping LIU, Wuxi (CN); Sen Zhao, Wuxi (CN)

(21) Appl. No.: 19/171,472

(22) Filed: Apr. 7, 2025

Related U.S. Application Data
(63) Continuation of application No. PCT/CN2024/128136, filed on Oct. 29, 2024.

(30) Foreign Application Priority Data
Apr. 19, 2024 (CN) 2024104743990

(51) Int. Cl. C12G 3/02; C12J 1/10

(52) U.S. Cl. CPC C12G 3/02; C12J 1/10

(57) ABSTRACT

The disclosure relates to an intelligent control system for a solid-state brewing producing process and its applications, belonging to the technical field of design and construction of devices for producing alcoholic beverages. The system includes a brewing container group, a storage device, a fed-batch system and a control system. An existing brewing pit is connected to the storage device, and a liquid collection groove, a pump body, a pressure gauge, a sensor, a spray recycling device and the like are additionally arranged to form an intelligent collection and circulation control system for fermented liquid products, thereby solving the technical problems of high labor intensity, low work efficiency, incomplete dripping of the products, and influence on the distillation efficiency of fermented grains caused by manual scooping of the products for recycling in the prior art, realizing automation of recycling the products, and facilitating the collection and recycling of the products.

### TECHNICAL FIELD

The disclosure relates to an intelligent control system for a solid-state brewing producing process and its applications, belonging to the technical field of design and construction of devices for producing alcoholic beverages.

### BACKGROUND

A fermented liquid product is free water produced through microbial metabolism of fermented grains and deposited at a bottom of a brewing container during a solid-state brewing process of food, which contains aromatic substances such as alcohols, acids and aldehydes, and contains beneficial microorganisms, saccharides, nitrogen-containing compounds and small amounts of tannins and pigments that are domesticated for a long time. During a fermentation process, the water, substances and microorganisms in the brewing container continuously migrate due to the leakage of fermented liquid products from top to bottom, so that fermented grains gradually become continuous and heterogeneous matrixes of substances and energy. Moreover, this heterogeneous situation continues to worsen with the extension of fermentation time, resulting in a significant difference in biological and physicochemical indexes of the upper, middle and lower layers of the fermented grains, thereby leading to a significant difference in quality of the brewed food. Furthermore, due to the important role of fermented liquid products in aging and curing of pit mud, this also leads to a significant difference in quality of pit mud at the bottom of the brewing container and at a lower layer of a pit wall and pit mud at middle and upper layers, thereby also affecting the quality of the brewed food produced from different levels of fermented grains.

Traditional fermented foods are produced by brewing processes in ancient China through microbial fermentation. There are many kinds of traditional fermented foods, which are mainly used for making wine, vinegar, sauce and soy sauce, and are also essential brewed condiments in Chinese life. In addition, fermented foods such as pickles, fermented bean curd and cheese are also popular traditional foods in China.

Taking Baijiu as an example, at present, major wine enterprises usually use fermented liquid products for re-steaming in a bottom pot, stirring of fermented grains, returning to a pit for fermentation, etc. During production of Baijiu, fermented liquid products are usually scooped by manual dripping, then transferred to destinations and collected for later use. This method has the problems of high labor intensity, low work efficiency, incomplete dripping of fermented liquid products, influence on the distillation efficiency of fermented grains, etc. Furthermore, traditional solid-state Baijiu fermentation relies heavily on pits, especially old pits, which greatly limits the increase of the yield of high-quality Baijiu of Baijiu enterprises. It is also an important technical problem for the Baijiu industry to make new pits mature as soon as possible and maintain stable microbial florae.

At present, there are few device systems for performing effective manual intervention and control on the flow of fermented liquid products in pits. In the prior art, there are single brewing and fermentation apparatuses only shown in CN112126540A, CN201722366U and CN116790329A, but the collection and storage of products thereof and the parameter control during a fermentation process still cannot be well automatically controlled, and require a significant amount of manpower for intervention and control. There is also an apparatus that only solves the problems of storage and collection shown in CN220149575U, but the apparatus is unable to link a front-end fermentation process to control various indexes and thus still has defects. Furthermore, during a process of sampling fermented liquid products, it is often unable to solve the technical problems of high labor intensity, low work efficiency, incomplete dripping of fermented liquid products, and influence on the distillation efficiency of fermented grains caused by manual scooping of fermented liquid products for recycling.

Therefore, there is an urgent need to find an apparatus that can automatically produce and collect fermented liquid products and control indexes thereof.

### SUMMARY

In order to solve the above problems, according to a first aspect, the disclosure provides an intelligent control system for a solid-state brewing producing process. The system includes:
a brewing container group, including at least three brewing containers connected in parallel, each of the brewing containers including a fermentation pit and a pit cover arranged at a top of the fermentation pit, a liquid outlet pipe being provided at a bottom of the fermentation pit, and a spray recycling device and a sampling groove being provided at a top of the pit cover;
a storage device circularly connected to the brewing container group, an outer surface of the storage device being at least provided with a feed port, a sampling port, a sight hole, a cooling water inlet, a cooling water outlet, a discharge port, an air inlet and a pollution emission port, and the inside of the storage device including a stirrer and a heating device;
a fed-batch system connected to the storage device; and
a control system connected to the brewing container group, the storage device and the fed-batch system,
where the brewing container group is circularly connected to the storage device, solid-state brewing is performed in the brewing container group, fermented liquid products after solid-state brewing flow into the storage device, then, at least a part of the reflux liquid in the storage device can be refluxed from the storage device to the spray recycling device of the brewing container group and uniformly sprayed to each brewing container for recycling, the fermented liquid products in the storage device can be detected in this process, and the solid-state fermentation process can be intelligently controlled through the fed-batch system and the control system according to detection situations. The reflux liquid is a substance in the storage device that has a certain viscosity and is rich in microorganisms, enzymes and the like, which can improve the subsequent fermentation quality.

Further, the brewing container is connected with at least one small liquid collection groove between the liquid outlet pipe and the pit cover, a bottom of the brewing container is connected to the liquid outlet pipe through a large liquid collection groove, and at least one sampling groove is further provided at the top of the pit cover. The large liquid collection groove and the small liquid collection groove are both connected to the storage device by an electric valve and a feed pipeline. The feed pipeline is provided with a pressure gauge, a pneumatic three-way ball valve and a feed pump. The small liquid collection groove is provided with an inner filter screen and an outer filter screen, and the large liquid collection groove is provided with a filter plate.

In some implementations of the disclosure, the liquid outlet pipe has a four-way pipe structure. An annular liquid groove is arranged at a periphery of a joint between the fermentation pit and the pit cover. The spray recycling device includes a plurality of concentric annular spray pipes mounted at the top of the pit cover, and each of the spray pipes is provided with a circulating liquid inlet.

In some implementations of the disclosure, the stirrer is a turbine stirrer with disc type flat blades and is provided with a defoamer, and the defoamer is provided with a rake type defoaming paddle.

In some implementations of the disclosure, the heating device includes an electric heating pipe arranged along an inner wall of the storage device, and the electric heating pipe is connected to a wiring box and a wiring hole on an outer wall of the storage device. The electric heating pipe has a vertical pipe bundle structure, and the wiring hole adopts a threaded surface.

In some implementations of the disclosure, an inner wall of the storage device is provided with a baffle plate. A top of the storage device includes a supplementing port correspondingly connected to each brewing container, a pressure gauge interface, a lamp-mirror assembly, at least one sensor socket and at least one sampling port, and the pressure gauge interface is connected with a pressure gauge.

In some implementations of the disclosure, the discharge port of the storage device is connected to the spray recycling device at the top of each pit cover through a discharge pipeline. The discharge pipeline is provided with a quick connector, a screw pump and a four-way valve.

Further, the fed-batch system includes a fed-batch tank and a constant flow pump connected to the fed-batch tank, and the constant flow pump is connected to the storage device.

In some implementations of the disclosure, a surface of the fed-batch tank is provided with a fed-batch port and a sight window, a bottom of the fed-batch tank is provided with a fed-batch discharge port, the fed-batch discharge port is connected to a needle valve, and the needle valve is connected to the constant flow pump.

According to a second aspect, the disclosure provides application of the intelligent control system for a solid-state brewing producing process in a brewing process of various flavored Baijiu (Chinese liquor), soy sauce, pickles, table vinegar, fermented soy beans, fermented bean curd, yogurt and miso.

The disclosure has the following beneficial effects:
(1) In the disclosure, an existing brewing pit is connected to the storage device, and the liquid collection groove, the pump body, the pressure gauge, the sensor, the spray recycling device and the like are additionally arranged to form an intelligent collection and circulation control system for fermented liquid products, thereby solving the technical problems of high labor intensity, low work efficiency, incomplete dripping of fermented liquid products, and influence on the distillation efficiency of fermented grains caused by manual scooping of fermented liquid products for recycling in the prior art, realizing an automation function of recycling fermented liquid products, and achieving the purpose of facilitating the collection and recycling of fermented liquid products. Moreover, the disclosure can control the indexes of fermented liquid products, indexes of spraying, and indexes in the pit through the fed-batch system, thereby achieving a better high-quality product rate.
(2) The spray recycling device provided by the disclosure can adopt a rectangular grid layout, which can improve the uniformity of drip irrigation. Moreover, the spray recycling device does not involve components such as a rotating motor and thus is easy to mount, and the air cannot enter the spray recycling device and affect fermentation, so that the spray recycling device is more suitable for traditional pit drip irrigation scenarios that use pit pressing soil for fermentation.
(3) The disclosure has relatively high precision in controlling the supplementing amount. By arranging the sight hole, supplementing port, pressure gauge interface, lamp-mirror assembly, sensor socket, sampling port and the like of the storage device, workers can clearly understand the remaining amount of nutrient substances in a storage tank.
(4) The disclosure can increase the content of ethyl caproate in Luzhou-flavor Baijiu by 23.37 mg/100 mL and increase the content of total acids by 0.08 g/L, and the proportions of ethyl caproate and ethyl lactate in Baijiu are more harmonious compared with a control group. The Baijiu yield is increased by 3.69%, the high-quality rate is increased by 12.92%, and thus, the effect is very significant.
(5) When acetic acid fermented liquid is added to Fen-flavor Baijiu through the disclosure, the content of ethyl acetate in Baijiu is increased by 88.43%, and the content of total esters is increased by 27.93%. In addition, the content of acetic acid in Baijiu is also increased, and the content of sour substances in Fen-flavor Baijiu is increased.
(6) In the disclosure, the Rhizopus esterifying enzyme technology is applied to production of soy sauce by a low-salt solid-state fermentation method, aiming to increase the content of ester flavor substances in soy sauce through an enzymatic method, which can significantly increase the content of volatile esters in basic soy sauce, reaching 7.918 mg/100 ml which is 39.3% higher than that in a control sample.
(7) In the disclosure, table vinegar with acidic protease can be added, which can increase the contents of alcohol and amino acid nitrogen, and achieve the optimal acidic protease activity with the highest alcohol content. At this time, the yield of alcohol is increased by 23.9%, and the content of amino acid nitrogen is increased by 240.7%.
(8) The disclosure can control more varieties and contents of flavor substances in pickles when the inoculation amount of high-degradation nitrite lactic acid bacteria is 0.375% and the inoculation amount of high-yield ester yeast is 0.5%, and 6 organic acids and 41 volatile flavor substances can be detected.

### BRIEF DESCRIPTION OF FIGURES

FIG. 1 is a schematic view of an overall structure in an implementation of the disclosure.

FIG. 2 is a structural cross-sectional view of a brewing container in an implementation of the disclosure.

FIG. 3 is a structural top view of a brewing container in an implementation of the disclosure.

FIG. 4 is a structural cross-sectional view of a spray recycling device in an implementation of the disclosure.

FIG. 5 is a structural cross-sectional view of a storage device in an implementation of the disclosure.

FIG. 6 is a structural top view of a storage device in an implementation of the disclosure.

FIG. 7 is a schematic structural view of a fed-batch system in an implementation of the disclosure.

In figures, 100: brewing container group, 200: stirrer, 300: heating device, 1: motor, 2: feed port, 3: cooling water outlet, 4: baffle plate, 5: stirring impeller, 6: sensor component, 6-1: sampling port, 6-2: first sensor socket, 6-3: second sensor socket, 6-4: third sensor socket, 7: air inlet, 8: pollution emission port, 9: discharge port, 10: cooling water inlet, 11: electric heating pipe, 12: wiring box, 13: wiring hole, 14: defoamer, 15: stirring shaft, 16: sight hole, 17: first supplementing port, 18: second supplementing port, 19: third supplementing port, 20: defoaming motor port, 21: pressure gauge interface, 22: lamp-mirror assembly, 23: spray recycling device, 24: pit cover, 25: annular liquid groove wall, 26: annular liquid groove, 27: fermentation pit, 28: inner filter screen, 29: small liquid collection groove, 30: filter plate, 31: liquid outlet pipe, 32: circulating liquid inlet, 33: sampling groove, 34: brewing container, 35: quick connector, 36: four-way valve, 37: screw pump, 38: pressure gauge, 39: pneumatic three-way ball valve, 40: electric valve, 41: large liquid collection groove, 42: filter hole, 43: manual ball valve, 44: filter plate, 45: storage device, 46: fed-batch port, 47: fed-batch tank, 48: fed-batch discharge port, 49: sight window, 50: constant flow pump, 51: needle valve, and 52: control system.

### DETAILED DESCRIPTION

The following clearly and completely describes the technical solutions in the embodiments of the present disclosure with reference to the accompanying drawings in the embodiments of the present disclosure. Apparently, the described embodiments are some of the embodiments of the present disclosure rather than all of the embodiments. All other embodiments obtained by those of ordinary skill in the art based on the embodiments of the present disclosure without creative work are within the protection scope of the present disclosure.

In the disclosure, unless otherwise clearly specified and defined, the terms “connected,” “connect” and “fix” should be broadly understood. For example, the connection may be fixed connection, detachable connection or integrated connection, may be mechanical connection or electrical connection, or may be direct connection, indirect connection through an intermediate medium, internal communication between two elements, or interaction relationship between two elements. For those of ordinary skill in the art, the specific meanings of the above terms in the disclosure can be understood according to specific situations.

In the disclosure, the “first” and “second” are only used to distinguish the same kind of components/parts with different positions or features, and have no other limiting meanings. The “up” refers to a direction in which each component deviates from the ground, and the “down” refers to a direction in which each component is away from the ground.

In the present disclosure, unless otherwise explicitly specified or defined, the first feature being “on” or “beneath” the second feature may include the first feature being in direct contact with the second feature or the first feature being in contact with the second feature through another feature rather than in direct contact with the second feature. In addition, the first feature being “on,” “above,” or “over” the second feature includes the first feature being right above or at the inclined top of the second feature, or merely indicates a level of the first feature being higher than that of the second feature. The first feature being “below,” “under,” and “beneath” the second feature includes the first feature being directly below and diagonally below the second feature, or simply indicates that the horizontal height of the first feature is less than that of the second feature.

### Embodiment 1

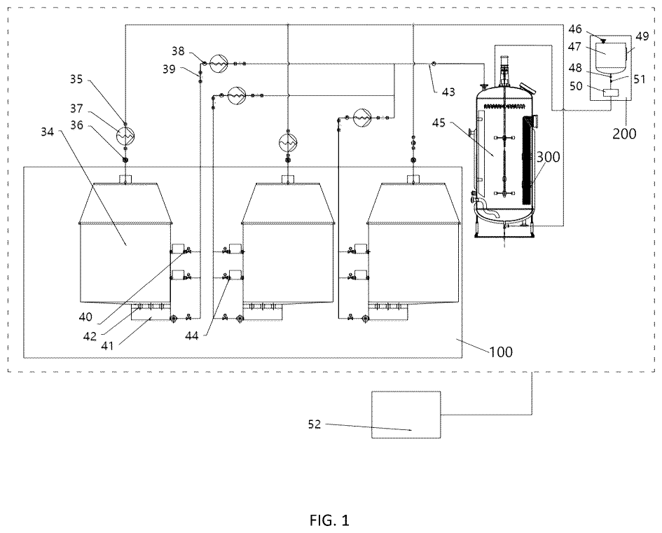

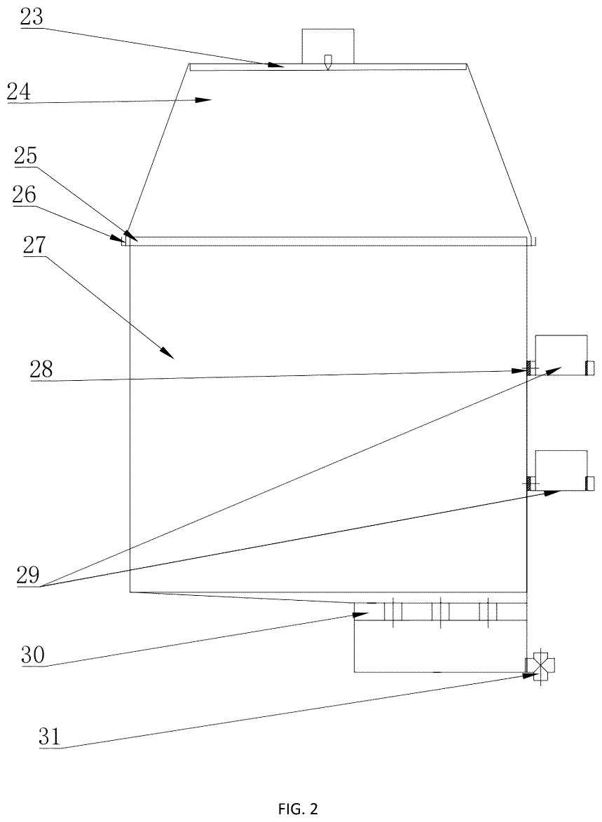

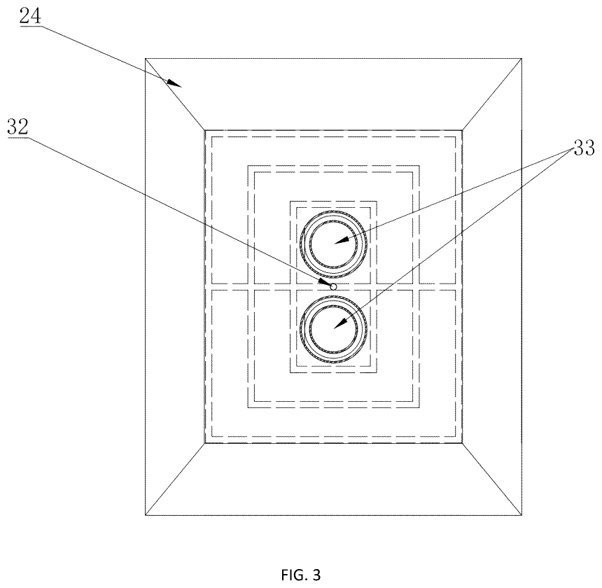

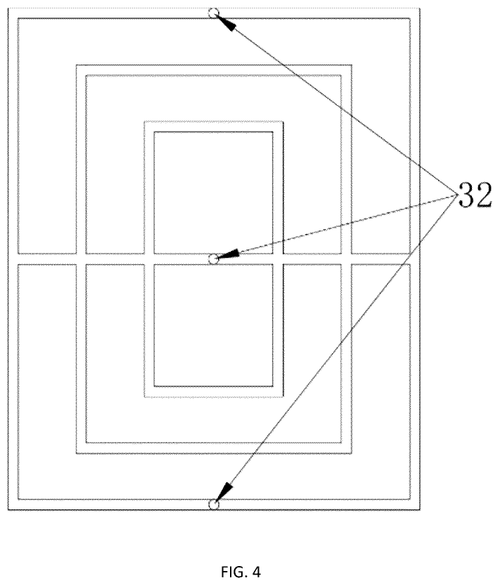

As shown in FIG. 1 to FIG. 4, the disclosure provides an intelligent control system for a solid-state brewing producing process. The system includes:
a brewing container group 100, including three brewing containers connected in parallel. The brewing container includes a fermentation pit 27 and a pit cover 24 arranged at a top of the fermentation pit 27. A liquid outlet pipe 31 is provided at a bottom of the fermentation pit 27. A spray recycling device 23 and a sampling groove 33 are provided at a top of the pit cover 24. The brewing container is connected with two small liquid collection grooves 29 at different heights between the liquid outlet pipe 31 and the pit cover 24. A bottom of the brewing container is connected to the liquid outlet pipe 31 through a large liquid collection groove 41. At least one sampling groove is further provided at the top of the pit cover 24. The liquid outlet pipe 31 has a four-way pipe structure, so that fermented liquid products can flow into different apparatuses. An annular liquid groove 26 is arranged at a periphery of a joint between the fermentation pit 27 and the pit cover 24. The spray recycling device 23 includes a plurality of concentric annular spray pipes and a sampling groove 33 mounted at the top of the pit cover 24, and each of the spray pipes is provided with a circulating liquid inlet 32.

The sampling groove 33 has a structure with a circular opening flip cover and a water groove connected around the flip cover, and is connected to the top of the pit cover 24 by welding mainly for sampling and achieving a sealing effect.

The large liquid collection groove 41 and the small liquid collection groove 29 are both connected to the storage device 45 by an electric valve 40 and a feed pipeline. The feed pipeline is provided with a pressure gauge 38, a pneumatic three-way ball valve 39 and a feed pump. The small liquid collection groove 29 is provided with an inner filter screen 28 and an outer filter screen 44, and the large liquid collection groove 41 is provided with a filter plate 30. A filter hole 42 is provided on one side of a bottom of the large liquid collection groove 41, and the large liquid collection groove 41 is connected to the liquid outlet pipe 31, thereby being convenient for controlling fermented liquid products of the large liquid collection groove 41 to enter and exit at any time. The inner filter screen 28, the outer filter screen 44 and the filter plate 30 are all configured to filter solid substances in fermented liquid products.

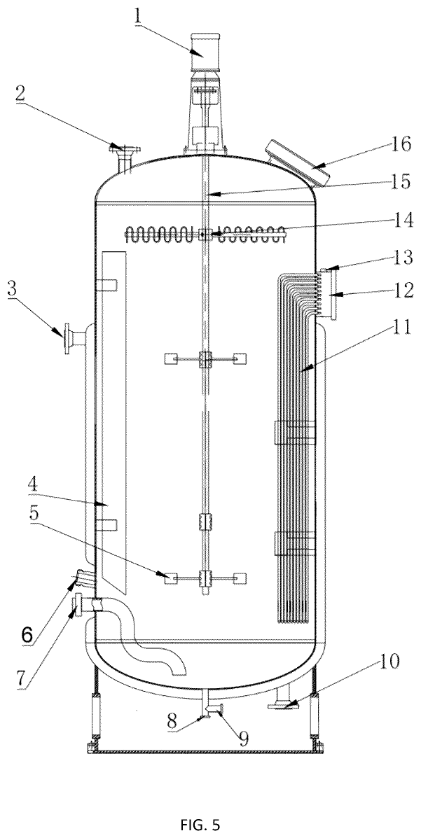

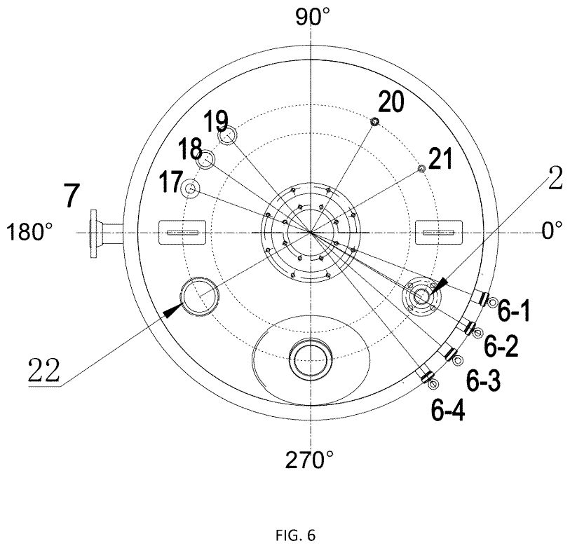

As shown in FIG. 5 to FIG. 6, the system further includes a storage device 45 circularly connected to the brewing container group 100. An outer surface of the storage device 45 is provided with a feed port 2, a sight hole 16, a cooling water inlet 10, a cooling water outlet 3, a discharge port 9, a sensor component 6, an air inlet 7 and a pollution emission port 8. The inside of the storage device 45 includes a stirrer 200 and a heating device 300. The stirrer 200 is a turbine stirrer with a stirring shaft 15 and a stirring impeller 5 and is provided with a defoamer 14. The stirring impeller 5 includes disc type flat blades. The defoamer 14 is provided with a rake type defoaming paddle. The heating device 300 includes electric heating pipes 11 arranged along an inner wall of the storage device 45. The electric heating pipes 11 are connected to a wiring box 12 and a wiring hole 13 on an outer wall of the storage device 45. The electric heating pipe 11 has a vertical pipe bundle structure. The wiring hole 13 adopts a threaded surface. The air is blown into the device through the air inlet 7 to achieve better uniformity. Furthermore, liquid heating and temperature control can be achieved through the electric heating pipe 11. Liquid parameters inside the storage device 45 can be monitored and controlled through a control system 52.

The inner wall of the storage device 45 is provided with a baffle plate 4. The baffle plate 4 has the functions of increasing the fluid turbulence, promoting the uniform distribution of oxygen, nutrients and microorganisms and improving the fermentation efficiency, can prevent solid particles in the fermented liquid from depositing at the bottom of the tank to avoid stratification and non-uniform fermentation of the fermented liquid, is favorable for dispersing the heat generated in the fermentation process, maintaining the temperature stability in the fermentation process and simultaneously preventing the overflow of excessive foam from the tank, and can promote the extraction of metabolites, improve the yield of products, promote mixing and metabolism and prevent precipitation and foam.

A top of the storage device 45 includes a first supplementing port 17, a second supplementing port 18 and a third supplementing port 19 which are correspondingly connected to each brewing container, a defoaming motor port 20, a pressure gauge interface 21, a lamp-mirror assembly 22, a first sensor socket 6-2, a second sensor socket 6-3, a third sensor socket 6-4 and a sampling port 6-1. The pressure gauge interface 21 is connected with a pressure gauge.

The dimension standards of the first supplementing port, the second supplementing port and the third supplementing port are all PBE 1″, and connecting surfaces are all hoops. The dimension standard of the defoaming motor port is RD28⅛, and a connecting surface is threaded and welded to the top of the tank to prevent the foam generated during fermentation or reaction from blocking the pipeline or overflowing. A connecting surface of the pressure gauge interface is threaded.

The feed port has a nominal dimension of DN25 and a connecting surface in the form of RF, and is connected to a tank body by welding. The cooling water inlet and the cooling water outlet have nominal dimensions of DN50 and connecting surfaces in the form of RF, and are welded to the tank body. The sampling port 6-1 is located on the same side and at the same height as the first sensor socket 6-2, the second sensor socket 6-3 and the third sensor socket 6-4, all of which are welded to the tank body, dimension standards are all R½, and connecting surfaces are all in threaded connection. The difference is that the sampling port 6-1 is vertically connected to the tank body, and an included angle between the sensor component 6 and the tank body is 75°. The purpose of obliquely arranging the sensor component 6 is to prevent the infiltration of the fermented liquid and protect the sensor from the risk of corrosion or short circuit. In addition, this design helps to maintain hygiene, reduce the accumulation of bacteria and microorganisms around the socket, and facilitate cleaning and disinfection. Furthermore, the bevel socket is convenient for mounting and maintenance, prevents misoperation, optimizes signal transmission, and reduces interference and damage caused by contact between a cable and the tank body. Therefore, this angle design ensures that the sensor can work safely and effectively and is also convenient for operation and maintenance, and the hygiene and stability of the fermentation process are maintained.

The air inlet is a single hole pipe ventilation device having a nominal dimension of DN50 and a connecting surface in the form of RF, and is welded to the tank body. The pollution emission port 8 and the discharge port 9 are integrated, and nominal dimensions are both DN32.

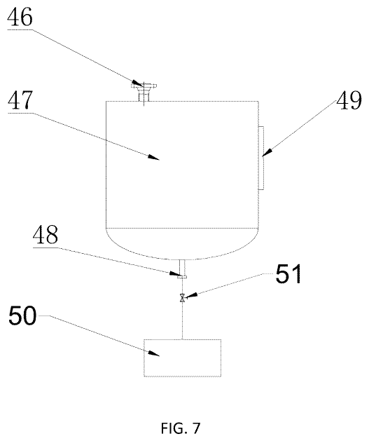

As shown in FIG. 7, the system further includes a fed-batch system connected to the storage device 45. The fed-batch system includes a fed-batch tank 47 and a constant flow pump 50 connected to the fed-batch tank 47. The constant flow pump 50 is connected to the storage device 45. A surface of the fed-batch tank 47 is provided with a fed-batch port 46 and a sight window 49. A bottom of the fed-batch tank 47 is provided with a fed-batch discharge port 48. The fed-batch discharge port 48 is connected to a needle valve 51. The needle valve 51 is connected to the constant flow pump 50. The constant flow pump 50 is connected to the first supplementing port 17, the second supplementing port 18 and the third supplementing port 19 of the storage device 45. The function of the needle valve 51 is to control the flow rate of liquid.

Furthermore, the system further includes a control system 52 connected to the brewing container group 100, the storage device 45 and the fed-batch system.

The brewing container group 100 is connected to the feed port 2 of the storage device 45 through a feed pipeline, and the discharge port 9 of the storage device 45 is connected to the spray recycling device 23 at the top of each pit cover through a discharge pipeline, thereby forming a circulating connection.

### Embodiment 2

The system in Embodiment 1 is used to perform solid-state brewing and intelligent collection and circulating control of fermented liquid products. The method is mainly as follows: solid-state brewing is performed in the brewing container group 100, fermented liquid products after solid-state brewing flow into the storage device 45, then, at least a part of the reflux liquid in the storage device 45 can be refluxed from the storage device 45 to the spray recycling device of the brewing container group 100 and uniformly sprayed to each brewing container for recycling, the fermented liquid products in the storage device 45 can be detected in this process, and the solid-state fermentation process can be intelligently controlled through the fed-batch system and the control system according to detection situations.

In this embodiment, the method specifically includes the following steps:
Step 1: Automatic extraction, collection and storage: Materials required for fermenting products are added to each brewing container for brewing and fermentation, and then, fermented liquid products are collected in the large liquid collection groove 41. Firstly, the fermented liquid products are extracted through a screw pump 37 on the feed pipeline and enter the storage device 45 through the feed port 2, and the flow rate and pressure may be controlled through the control system 52, the pneumatic three-way ball valve 39, the electric valve 40 and the pressure gauge 38 in this process. Secondly, the fermented liquid products at middle and upper layers, collected by the small liquid collection groove 29, may also be extracted through the screw pump 37 on the feed pipeline and enter the storage device 45 through the feed port 2, and the flow rate, pH and pressure may be controlled through the control system 52, the pneumatic three-way ball valve 39, the electric valve 40, the pressure gauge 38, the constant flow pump 50 and the needle valve 51 in this process.
Step 2: The in-tank pressure, capacity, oxygen content, pH and temperature indexes inside the storage device 45 are monitored through the control system 52, the pressure gauge interface 21, the lamp-mirror assembly 22, the first sensor socket 6-2, the second sensor socket 6-3, the third sensor socket 6-4 and the sampling port 6-1, thereby further adjusting the pneumatic three-way ball valve 39, the electric valve 40, the pressure gauge 38, the constant flow pump 50 and the needle valve 51 according to the indexes, so as to obtain in-tank pressure, filling level, dissolved oxygen content, pH and temperature information.
Step 3: When it is judged that the fermented liquid products in the storage device 45 reach a lower end of the defoamer 14 through the lamp-mirror assembly 22, a motor 1 in the storage device 45 is turned on for stirring to drive the stirring shaft 15 and the stirring impeller 5 to uniformly stir the liquid and disperse bubbles; simultaneously, the air inlet 7 is opened for blowing air to uniformly mix the liquid; then, the defoamer 14 is turned on for eliminating bubbles to prevent the bubbles from overflowing from the top of the tank; and finally, the electric heating pipe 11 is turned on or the cooling water inlet 10 is opened for heating or cooling to maintain a certain temperature.
Step 4: Recycling: The reflux liquid rich in microorganisms, enzymes and other substances that improve the subsequent fermentation quality in the storage device 45 is extracted from the discharge port 9 through the screw pump 37 on the discharge pipeline after being controlled by the fed-batch system, then is pumped into the spray recycling device 23 at the top of each brewing container, enters the spray pipe through each circulating liquid inlet 32, and is uniformly sprayed to the inside of the brewing container through the spray pipe, thereby forming recycling.

### Embodiment 3: Control of Luzhou-Flavor Solid-State Baijiu Fermentation

The system in Embodiment 1 and the method in Embodiment 2 were used.

Before operation, esterified Monascus enzyme, edible alcohol (the ethanol content was 15%-20%), composite organic acids and the like were added to the fed-batch tank 47 through the fed-batch port 46.

In step 1, sorghum serving as a main raw material was added to the brewing container, and Daqu was cultured at a medium temperature; and Daqu was prepared from barley and wheat as raw materials and cultured with a certain proportion of peas for fermentation. In step 2, the pH needed to be adjusted to 5-6. In step 3, the temperature needed to be adjusted to the index of 35° C.; and the fed-batch system might use the fed-batch esterified red yeast to produce yellow water esterification liquid. In step 4, the yellow water in the storage device 45 was extracted from the discharge port 9 to the spray recycling device 23 for recycling and uniform spraying.

Finally, the content of ethyl caproate in Luzhou-flavor Baijiu could be increased by 23.37 mg/100 mL, the content of total acids could be increased by 0.08 g/L, and the proportions of ethyl caproate and ethyl lactate in Baijiu were more harmonious compared with a control group. Moreover, the Baijiu yield was increased by 3.69%, the high-quality rate was increased by 12.92%, and thus, the effect was very significant.

### Embodiment 4: Control of Fen-Flavor Solid-State Baijiu Fermentation

The system in Embodiment 1 and the method in Embodiment 2 were used.

Before operation, low-yield n-propanol yeast liquid was added to the fed-batch tank 47 through the fed-batch port 46.

In step 1, sorghum, fresh materials, vinasse, Daqu, auxiliary materials and water were added to the brewing container and mixed uniformly in proportion for fermentation. In step 2, the pH needed to be adjusted to the index of 3-8. In step 3, the temperature needed to be adjusted to the index of 20-35° C., the content of glucose was 2%-25%, and the volume fraction of ethanol was 0-12%; and the fed-batch system might fed-batch feed the low-yield n-propanol yeast liquid to the storage device 45. In step 4, the reflux liquid in the storage device 45 was extracted from the discharge port 9 to the spray recycling device 23 for recycling and uniform spraying.

Finally, the content of ethyl acetate in Baijiu was increased by 88.43%, and the content of total esters was increased by 27.93%. In addition, the content of n-propanol was significantly reduced by 29.9%, the Baijiu yield (34.85%) was increased, the total content of isobutanol, isopentanol and fusel oil (including the contents of n-propanol, isobutanol and isopentanol) were all reduced by 6.6%, 4.4% and 11.7% respectively, and the content of sour substances in Fen-flavor Baijiu was increased.

### Embodiment 5: Control of Production of Soy Sauce by Low-Salt Solid-State Fermentation Method

The system in Embodiment 1 and the method in Embodiment 2 were used.

Before operation, Rhizopus esterification enzyme was added to the fed-batch tank 47 through the fed-batch port 46.

In step 1, bean meal, wheat flour and bran were added to the brewing container in a ratio of 6:2:2, and 1.3 times of water and yeast were added for fermentation. In step 2, the pH needed to be adjusted to the index of 5-7. In step 3, the temperature needed to be adjusted to the index of 45° C.; and the fed-batch system might fed-batch feed the Rhizopus esterification enzyme to the storage device 45, and the content was controlled at 1%. In step 4, the reflux liquid in the storage device 45 was extracted from the discharge port 9 to the spray recycling device 23 for recycling and uniform spraying.

Finally, the content of ester flavor substances in soy sauce could be increased, and the content of volatile esters in basic soy sauce could be significantly increased to 7.918 mg/100 ml which was increased by 39.3%.

### Embodiment 6: Control of Mature Vinegar

The system in Embodiment 1 and the method in Embodiment 2 were used.

Before operation, Aspergillus niger acidic protease was added to the fed-batch tank 47 through the fed-batch port 46.

In step 1, edible corn starch, bran, rice hulls, bacterial inhibitors, table vinegar koji extract, edible salt, sodium benzoate and water for production were added to the brewing container for fermentation. In step 2, the pH needed to be adjusted to the index of 2-5. In step 3, the temperature needed to be adjusted to the index about 45° C.; and the fed-batch system might fed-batch feed the Aspergillus niger acidic protease to the storage device 45. In step 4, the reflux liquid in the storage device 45 was extracted from the discharge port 9 to the spray recycling device 23 for recycling and uniform spraying.

Finally, the contents of alcohol and amino acid nitrogen could be increased, and the optimal acidic protease activity with the highest alcohol content could be achieved. At this time, the yield of alcohol was increased by 23.9%, and the content of amino acid nitrogen was increased by 240.7%, thereby improving the flavor and quality of mature vinegar.

### Embodiment 7: Control of Fermentation of Pickles

The system in Embodiment 1 and the method in Embodiment 2 were used.

Before operation, a bamboo leaf flavone solution was added to the fed-batch tank 47 through the fed-batch port 46.

In step 1, Chinese cabbage, salt, sugar, vinegar, sesame oil, dried chili, fistular onion stalk, ginger and the like were added to the brewing container for fermentation. In step 2, the pH needed to be adjusted to the index of 4-5. In step 3, the temperature needed to be adjusted to the index about 15° C.; and the fed-batch system might fed-batch feed the bamboo leaf flavone solution to the storage device 45. In step 4, the reflux liquid in the storage device 45 was extracted from the discharge port 9 to the spray recycling device 23 for recycling and uniform spraying.

Finally, this embodiment could control more varieties and contents of flavor substances in pickles when the inoculation amount of high-degradation nitrite lactic acid bacteria was 0.375% and the inoculation amount of high-yield ester yeast was 0.5%, and 6 organic acids and 41 volatile flavor substances could be detected.

Although the present disclosure has been disclosed as above in exemplary examples, it is not intended to limit the present disclosure. Those skilled in the art can make various changes and modifications without departing from the spirit and scope of the present disclosure. Therefore, the protection scope of the present disclosure shall be as defined in the Claims.

What is claimed is:

1. An intelligent control system for a solid-state brewing producing process, comprising:
a brewing container group (100), comprising at least three brewing containers connected in parallel, each of the brewing containers comprising a fermentation pit (27) and a pit cover (24) arranged at a top of the fermentation pit (27), a liquid outlet pipe (31) being provided at a bottom of the fermentation pit (27), and a spray recycling device (23) and a sampling groove (33) being provided at a top of the pit cover (24);
a storage device (45) circularly connected to the brewing container group (100), an outer surface of the storage device (45) being at least provided with a feed port (2), a sampling port (6-1), a sight hole (16), a cooling water inlet (10), a cooling water outlet (3), a discharge port (9), an air inlet (7) and a pollution emission port (8), and the inside of the storage device (45) comprising a stirrer (200) and a heating device (300);
a fed-batch system connected to the storage device (45); and
a control system (52) connected to the brewing container group (100), the storage device (45) and the fed-batch system,
wherein the brewing container group (100) is circularly connected to the storage device (45), solid-state brewing is performed in the brewing container group (100), fermented liquid products after solid-state brewing flow into the storage device (45), then, at least a part of the reflux liquid in the storage device (45) is configured to be refluxed from the storage device (45) to the spray recycling device (23) of the brewing container group (100) and uniformly sprayed to each brewing container for recycling, the fermented liquid products in the storage device (45) are to be detected in this process, and the solid-state fermentation process is intelligently controlled through the fed-batch system and the control system (52) according to detection situations.

2. The intelligent control system for a solid-state brewing producing process according to claim 1, wherein the brewing container is connected with at least one small liquid collection groove (29) between the liquid outlet pipe (31) and the pit cover (24), a bottom of the brewing container is connected to the liquid outlet pipe (31) through a large liquid collection groove (41), and at least one sampling groove is further provided at the top of the pit cover (24).

3. The intelligent control system for a solid-state brewing producing process according to claim 2, wherein the large liquid collection groove (41) and the small liquid collection groove (29) are both connected to the storage device (45) by an electric valve (40) and a feed pipeline.

4. The intelligent control system for a solid-state brewing producing process according to claim 3, wherein the feed pipeline is provided with a pressure gauge (38), a pneumatic three-way ball valve (39) and a feed pump.

5. The intelligent control system for a solid-state brewing producing process according to claim 3, wherein the small liquid collection groove (29) is provided with an inner filter screen (28) and an outer filter screen (44), and the large liquid collection groove (41) is provided with a filter plate (30).

6. The intelligent control system for a solid-state brewing producing process according to claim 3, wherein the liquid outlet pipe (31) has a four-way pipe structure.

7. The intelligent control system for a solid-state brewing producing process according to claim 3, wherein an annular liquid groove (26) is arranged at a periphery of a joint between the fermentation pit (27) and the pit cover (24).

8. The intelligent control system for a solid-state brewing producing process according to claim 3, wherein the spray recycling device (23) comprises a plurality of concentric annular spray pipes mounted at the top of the pit cover (24), and each of the spray pipes is provided with a circulating liquid inlet (32).

9. The intelligent control system for a solid-state brewing producing process according to claim 3, wherein the stirrer (200) is a turbine stirrer with disc type flat blades and is provided with a defoamer (14), and the defoamer (14) is provided with a rake type defoaming paddle.

10. The intelligent control system for a solid-state brewing producing process according to claim 3, wherein the heating device (300) comprises an electric heating pipe (11) arranged along an inner wall of the storage device (45), and the electric heating pipe (11) is connected to a wiring box (12) and a wiring hole (13) on an outer wall of the storage device (45).

11. The intelligent control system for a solid-state brewing producing process according to claim 10, wherein the electric heating pipe (11) has a vertical pipe bundle structure, and the wiring hole (13) adopts a threaded surface.

12. The intelligent control system for a solid-state brewing producing process according to claim 1, wherein an inner wall of the storage device (45) is provided with a baffle plate (4).

13. The intelligent control system for a solid-state brewing producing process according to claim 1, wherein a top of the storage device (45) comprises a supplementing port correspondingly connected to each brewing container, a pressure gauge interface (21), a lamp-mirror assembly (22), at least one sensor socket and at least one sampling port, and the pressure gauge interface (21) is connected with a pressure gauge.

14. The intelligent control system for a solid-state brewing producing process according to claim 1, wherein a discharge port (9) of the storage device (45) is connected to the spray recycling device (23) at the top of each pit cover through a discharge pipeline.

15. The intelligent control system for a solid-state brewing producing process according to claim 14, wherein the discharge pipeline is provided with a quick connector (35), a screw pump (37) and a four-way valve (36).

16. The intelligent control system for a solid-state brewing producing process according to claim 1, wherein the fed-batch system comprises a fed-batch tank (47) and a constant flow pump (50) connected to the fed-batch tank (47), and the constant flow pump (50) is connected to the storage device (45).

17. The intelligent control system for a solid-state brewing producing process according to claim 16, wherein a surface of the fed-batch tank (47) is provided with a fed-batch port (46) and a sight window (49), a bottom of the fed-batch tank (47) is provided with a fed-batch discharge port (48), the fed-batch discharge port (48) is connected to a needle valve (51), and the needle valve (51) is connected to the constant flow pump (50).
